(19) United States
(12) Patent Application Publication
(10) Pub. No.: US 2020/0345594 A1
(43) Pub. Date: Nov. 5, 2020

(54) GELATINOUS COMPOSITION AND PRODUCTION METHOD THEREFOR

(71) Applicant: KANEKA CORPORATION, Osaka (JP)

(72) Inventor: Tadao Tsuji, Osaka (JP)

(73) Assignee: KANEKA CORPORATION, Osaka (JP)

(21) Appl. No.: 16/923,937

(22) Filed: Jul. 8, 2020

Related U.S. Application Data
(63) Continuation of application No. PCT/JP2018/044679, filed on Dec. 5, 2018.

(30) Foreign Application Priority Data
Jan. 12, 2018 (JP) 2018-003099

(51) Int. Cl. A61K 8/04; A61K 8/19; A61K 8/64; A61K 8/34; A61Q 19/00; A61K 8/41; A61K 8/49; A61K 8/44

(52) U.S. Cl. CPC A61K 8/042; A61K 8/19; A61K 8/64; A61K 8/345; A61K 2800/805; A61K 8/41; A61K 8/4913; A61K 8/44; A61Q 19/00

(57) ABSTRACT

A gelatinous composition may include a biosurfactant, water and/or a polyvalent alcohol, an oleaginous component, and an alkaline substance. The alkaline substance may be one or more selected from the group consisting of a hydroxy group-containing amine compound, an alkali metal hydroxide, and a basic amino acid. A concentration of the oleaginous component may be 50 to 99 mass %.

### TECHNICAL FIELD

One or more embodiments of the present disclosure relate to a gelatinous composition excellent in stability over time and a method for producing a gelatinous composition excellent in stability over time.

### BACKGROUND

Various dosage forms of cosmetics have been developed. The example of such dosage forms includes a gelatinous composition prepared by increasing the viscosity of an oleaginous component. A gelatinous composition is applied to various skin preparations and cosmetic preparations, such as cleansing cosmetics and hair cosmetics. A composition containing a polyvalent alcohol and a surfactant has been conventionally known as such a gelatinous composition prepared by increasing the viscosity of an oleaginous component. For example, a gelatinous composition containing surfactin as a biosurfactant, an analogous compound thereof or a salt thereof and a trivalent or more polyvalent alcohol is known (Patent document 1). A gelatinous composition may be compatible with skin and give a smooth feeling during use, but may not exhibit stability over time. For example, when a gelatinous composition is preserved in a gel state for a long time, an oleaginous component may become separated.

In addition, an exothermic composition and calefacient cosmetics which may contain a biosurfactant is known (Patent document 2), and a skin preparation containing surfactin as a biosurfactant is known (Patent document 3).

Patent Document 1: JP 2003-176211 A

Patent Document 2: JP 2004-131413 A

Patent Document 3: JP 2004-67647 A

### SUMMARY

One or more embodiments of the present disclosure provide a gelatinous composition excellent in stability over time, a skin preparation and a cosmetic preparation which contain the gelatinous composition, and a method for producing a gelatinous composition excellent in stability over time.

In one or more embodiments, a gelatinous composition excellent in preservation stability can be obtained by adding the specific alkaline substance to a gelatinous composition containing a biosurfactant and a polyvalent alcohol.

Hereinafter, one or more embodiments of the present disclosure is described.

[1] A gelatinous composition,

comprising a biosurfactant, water and/or a polyvalent alcohol, an oleaginous component, and an alkaline substance,

wherein the alkaline substance is one or more selected from a hydroxy group-containing amine compound, an alkali metal hydroxide and a basic amino acid, and

wherein a concentration of the oleaginous component is 50 mass % or more and 99 mass % or less.

[2] The gelatinous composition according to the above [1], wherein the hydroxy group-containing amine compound is one or more selected from triethanolamine, diethanolamine, monoethanolamine, diisopropanolamine, triisopropanolamine, 2-amino-2-methylpropanol, 2-amino-2-methylpropanediol, 2-amino-2-ethyl-1,3-propanediol, 2-amino-2-hydroxymethyl-1,3-propanediol and DL-1 amino-2-propanol.

[3] The gelatinous composition according to the above [1] or [2] , wherein the alkali metal hydroxide is one or more selected from NaOH and KOH.

[4] The gelatinous composition according to any one of the above [1] to [3], wherein the biosurfactant is one or more selected from surfactin, arthrofactin, iturin, and salts thereof.

[5] The gelatinous composition according to any one of the above [1] to [4], wherein the polyvalent alcohol is one or more selected from glycerin, sorbitol, xylitol, diglycerin and polyethyleneglycol.

[6] The gelatinous composition according to any one of the above [1] to [5], wherein pH of the gelatinous composition is 7.0 or more and 11.0 or less.

[7] A skin preparation, comprising the gelatinous composition according to any one of the above [1] to [6].

[8] A cosmetic preparation, comprising the gelatinous composition according to any one of the above [1] to [6].

[9] A method for producing a gelatinous composition,

wherein the gelatinous composition comprises a biosurfactant, water, an alkaline substance, a polyvalent alcohol, and an oleaginous component,

wherein the alkaline substance is one or more selected from a hydroxy group-containing amine compound, an alkali metal hydroxide and a basic amino acid, and

wherein a concentration of the oleaginous component is 50 mass % or more and. 99 mass % or less,

comprising the steps of:

dissolving the biosurfactant and the alkaline substance in all or a part of the water to obtain a first solution,

mixing the first solution with the polyvalent alcohol to obtain a second solution, and

adding the oleaginous component or the remnant water and the oleaginous component to the second solution.

[10] The method according to the above [9], wherein the hydroxy group-containing amine compound is one or more selected from triethanolamine, diethanolamine, monoethanolamine, diisopropanoiamine, triisopropanoiamine, 2-amino-2-methylpropanol, 2-amino-2-methylpropanediol, 2-amino-2-ethyl-1,3-propanediol, 2-amino-2-hydroxymethyl-1,3-propanediol and DL-1-amino-2-propanol.

[11] The method according to the above [9] or [10], wherein the alkali metal hydroxide is one or more selected from NaOH and KOH.

[12] The method according to any one of the above [9] to [11], wherein the biosurfactant is one or more selected from a surfactin salt, an arthrofactin salt and an iturin salt.

[13] The method according to any one of the above [9] to [12], wherein the polyvalent alcohol is one or more selected from glycerin, sorbitol, xylitol, diglycerin and polyethyleneglycol.

[14] The method according to any one of the above [9] to [13], wherein pH of the gelatinous composition is 7.0 or more and 11.0 or less.

### DETAILED DESCRIPTION

A gelatinous composition excellent in stability over time can be obtained according to one or more embodiments of the present disclosure.

The gelatinous composition of one or more embodiments of the present disclosure contains a biosurfactant. A biosurfactant is a natural compound which is produced by a microorganism. In general, a biosurfactant is characterized by extremely high safety to the environment and a human body, since a biosurfactant is highly biodegradable and has low skin irritation to a human body. The biosurfactant usable in one or more embodiments of the present disclosure is exemplified by a lipopeptide compound biosurfactant such as surfactin, arthrofactin and iturin; a glycolipid biosurfactant such as mannosylerythritol lipid, sophorolpid, trehalose lipid and rhamnolipid; a fatty acid biosurfactant such as spiculisporic acid; a polymer biosurfactant such as emulsan; and salts thereof, and is not restricted thereto.

Among the above examples, a lipopeptide biosurfactant as a lipopeptide compound may be preferred, surfactin, arthrofactin, iturin or salts thereof as a cyclic lipopeptide biosurfactant is provided, and surfactin or a salt thereof is provided in one or more embodiments. The gelatinous composition containing a cyclic lipopeptide biosurfactant is provided in one or more embodiments, since such a gelatinous composition tends to give a smooth feeling without rough feeling nor stickiness.

A salt of surfactin means the compound represented by the formula (1) or a composition containing two or more kinds of the compounds in one or more embodiments of this disclosure.

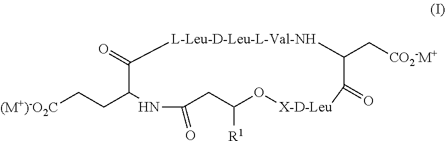

wherein

‘X’ is a residue of an amino acid selected from leucine, isoleucine and valine;

R¹ is a C₉₋₁₈ alkyl group;

‘M⁺’ is an alkali metal ion or a quaternary ammonium ion.

Although the amino acid residue as ‘X’ may be either in an L-form or a D-form; the L-form is provided in one or more embodiments.

The term “C₉₋₁₈ alkyl group” means a linear or branched monovalent saturated hydrocarbon group having 9 or more and 18 or less carbon atoms. An example thereof includes n-nonyl, 6-methyloctyl, 7-methyloctyl, n-decyl, 8-methylnonyl, n-undecyl, 9-methyldecyl, n-dodecyl, 10-methylundecyl, n-tridecyl, 11-methyldodecyl, n-tetradecyl, n-pentadecyl, n-hexadecyl, n-heptadecyl and n-octadecyl.

The alkali metal ion is not particularly restricted, exemplified by a lithium ion, a sodium ion, a potassium ion, and a sodium ion, or any other suitable ion.

The example of a substituent of the quaternary ammonium ion includes an organic group, for example, an alkyl group such as methyl, ethyl, n-propyl, isopropyl, n-butyl and tert-butyl; an aralkyl group such as benzyl, methylbenzyl and phenyiethyl; and an aryl group such as phenyl, toluyl and xylyl. An example of the quaternary ammonium ion includes a tetramethylammonium ion, a tetraethylammonium ion and a pyridinium ion.

Arthrofactin is represented by the formula (II).

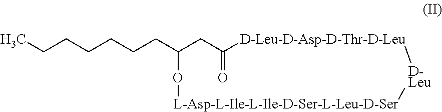

Arthrofactin has one D-aspartic acid and one L-aspartic acid respectively in the structure, and may form a salt with an alkali metal ion or a quaternary ammonium ion.

Iturin is represented by the formula (III).

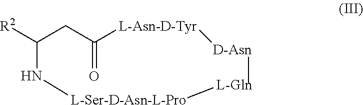

In the formula (III), R² is a C₉₋₁₈ alkyl group such as —(CH₂)₁₀CH₃, —(CH₂)₈CH (CH₃) CH₂CH₃ and —(CH₂)₉CH (CH₃)₂.

One kind or two or more kinds of biosurfactants or salts thereof may be used.

The biosurfactant or salt thereof can be obtained by cultivating a microorganism which produces the target biosurfactant and separating the biosurfactant or salt thereof from a culture liquid of the microorganism in accordance with a conventional method. The purified biosurfactant may be used or the unpurified biosurfactant such as a culture liquid may be used. An example of a microorganism which produces surfactin includes a strain classified in Bacillus subtilis. The biosurfactant which is chemically synthesized can be also used similarly.

For example, a concentration of the biosurfactant in the gelatinous composition according to one or more embodiments of the present disclosure can be adjusted to 0.02 mass % or more and 3 mass % or less. When the concentration is 0.02 mass % or more, the stability over time of the gelatinous composition can be ensured more surely. When the concentration is 3 mass % or less, the deterioration of use feeling due to the excessive biosurfactant can be prevented more surely. The above concentration may be 0.05 mass % or more or 0.1 mass % or more, and 2 mass % or less or 1.5 mass % or less.

The gelatinous composition of one or more embodiments of the present disclosure contains water and/or a polyvalent alcohol. The water and/or polyvalent alcohol means at least one selected from water and a polyvalent alcohol or one or more selected from water and a polyvalent alcohol, and the gelatinous composition of one or more embodiments of the present disclosure may contain both of water and a polyvalent alcohol. The polyvalent alcohol is not particularly restricted and one or more selected from glycerin, sorbitol, xylitol, diglycerin, and polyethyleneglycol may be used.

For example, a concentration of the water and/or polyvalent alcohol in the gelatinous composition of one or more embodiments of the present disclosure can be adjusted to 1 mass % or more and 50 mass % or less or 1.5 mass % or more and 30 mass % or less. When the concentration of the water and/or polyvalent alcohol is included in the above range, the gelatinous composition with excellent use feeling can be produced.

When both of the water and polyvalent alcohol are used, a ratio thereof may be appropriately adjusted. For example, a ratio of the water to the total of the water and polyvalent alcohol can be adjusted to 40 mass % or more and 95 mass % or less, or 50 mass % or more and 90 mass % or less.

The gelatinous composition of one or more embodiments of the present disclosure contains an oleaginous component. The oleaginous component of one or more embodiments of the present disclosure is not particularly restricted as long as the oleaginous component is not mixed with water at an arbitrary ratio. More specifically, the gelatinous component means a substance which is not dissolved within 30 minutes when the substance is added to 1000 mL or more of water and the mixture is strongly shaken to be mixed at 20±5° C. for 30 seconds every 5 minutes. In one or more embodiments, it is provided to use one kind or two or more kinds selected from a hydrocarbon such as squalane, liquid paraffin, light liquid paraffin, ceresin, polyethylene powder, squalene, microcrystalline wax, vaseline, liquid isoparaffin, polybutene and mineral oil; wax such as beeswax, carnauba wax, candelilla wax, jojoba oil, lanolin and spermaceti; a fat and oil, such as macadamia nut oil, olive oil, cottonseed oil, soybean oil, avocado oil, rice bran oil, rice oil, rice germ oil, palm kernel oil, castor oil, rosehip oil, evening primrose oil, camellia oil, horse oil, grape seed oil, palm oil, meadowfoam seed oil, shea butter, corn oil, safflower oil and sesame oil; an ester such as echylhexyl palmitate, isononyl isononanoate, isopropyl myristate, ethyl oleate, glyceryl tri(caprylate/caprate), cetyl 2-ethylhexanoate, glyceryl tri(2-ethylhexanoate), diisopropyl sebacate and cholesteryl hydxoxystearate; a fatty acid such as myristic acid, stearic acid and oleic acid; a silicone oil such as methylpolysiloxane, methylphenylpolysiloxane and amino-modified silicone; a higher alcohol such as cetanol and oleyl alcohol; and an alkyl glyceryl ether such as batyl alcohol and chimyl alcohol.

A concentration of the oleaginous component in the gelatinous composition of one or more embodiments of the present disclosure may be adjusted to, for example, 50 mass % or more and 99 mass % or less. The concentration may be 51 mass % or more, or 70 mass % or more and 95 mass % or less. When the concentration of the oleaginous component is included in the above range, the gelatinous composition with. excellent use feeling can be obtained.

The gelatinous composition of one or more embodiments of the present disclosure contains an alkaline substance. The alkaline substance may be one or more selected from a hydroxy group-containing amine compound, an alkali metal hydroxide and a basic amino acid.

The hydroxy group-containing amine compound of one or more embodiments may be triethanolamine, diethanolamine, monoethanolamine, diisopropanolamine, triisopropanolamine, 2-amino-2-methylpropanol, 2-amino-2-methylpropanediol, 2-amino-2-ethyl 1,3 propanediol, 2-amino-2-hydroxymethyl-1,3-propanediol or DL-1-amino-2-propanol. The hydroxy group-containing amine compound of one or more embodiments may be triethanolamine, diethanolamine, monoethanolamine, diisopropanolamine, triisopropanolamine, 2-amino-2-methylpropanol, 2-amino-2-methylpropanediol, 2-amino-2-ethyl-1,3-propanediol or DL-1-amino-2-propanol.

The alkali metal hydroxide of one or more embodiments may be NaOH or KOH.

The basic amino acid of one or more embodiments may be arginine, histidine, or lysine.

A concentration of the alkaline substance in the gelatinous composition of one or more embodiments of the present disclosure may be adjusted to, for example, 0.001 mass % or more and 2 mass % or less. The concentration may be 0.005 mass % or more, and 1 mass % or less, or 0.5 mass % or less. When the concentration of the alkaline substance is included in the above range, the gelatinous composition is stabilized.

Some amine compounds classified in the alkaline substance may form a salt with the biosurfactant which is an anionic surfactant; therefore, in one or more embodiments it is provided to use a biosurfactant salt as the biosurfactant and to use the alkaline substance in addition to the biosurfactant salt for producing the gelatinous composition.

An optional component may be added to the gelatinous composition of one or more embodiments of the present disclosure as long as the effect of one or more embodiments of the present disclosure is achieved. An example of such an optional component includes a lower alcohol such as ethanol and isopropanol; an anionic surfactant, a cationic surfactant, an amphoteric surfactant, a thickener, an ultraviolet absorber, an antioxidant, an emollient agent, an emulsifier, a solubilizer, an anti-inflammatory drug, a humectant, a preservative, a disinfectant, a dye, a fragrance and a powder. It is provided not to add a mineral such as zeolite to the gelatinous composition of one or more embodiments of the present disclosure.

When the optional component is used, a concentration of the optional component in the emulsion composition of one or more embodiments of the present disclosure may be adjusted depending on a kind of the optional component or the like and may be adjusted to, for example, 0.01 mass % or more and 10 mass % or less.

The pH of the gelatinous composition according to one or more embodiments of the present disclosure may be adjusted to, for example, 1.0 or more and 11.0 or less or 7.5 or more and 10.0 or less. The stability of the gelatinous composition can be expected to be further improved by adjusting the pH of the gelatinous composition to the above-described range.

A viscosity of the gelatinous composition according to one or more embodiments of the present disclosure may be appropriately adjusted and may be adjusted to, for example, 10,000 mPa·s or more. The upper limit of the viscosity is not particularly restricted, and the viscosity may be adjusted to, for example, 100,000 mPa·s or less.

The gelatinous composition of one or more embodiments of the present disclosure can be produced by, for example, dissolving the biosurfactant and the alkaline substance in the water and/or polyvalent alcohol and adding the oleaginous component to the stirred mixture in small batches. When water and the polyvalent alcohol are used in combination, all or a part of the water may be added after the oleaginous component is added.

The oleaginous component may be added by a predetermined volume or in a continuous manner. Adding by a predetermined volume is referred to as divided addition, and adding in a continuous manner is referred to as continuous addition. In a case of the divided addition, 60 mass % or less of the oleaginous component to the amount of the already added water and/or polyvalent alcohol is added at one time, and the mixture is stirred to be homogenous. The above ratio may be 30 mass % or less, or 10 mass % or less. The required amount of the oleaginous component is added by repeating this procedure. In a case of the continuous addition, an addition rate may be adjusted to 60 mass % or less of the amount of the already added water and/or polyvalent alcohol per minute. The addition rate may be 30 mass %/min or less or 10 mass %/min or less.

When the optional component is added, the optional component may be added by any methods; for example, the optional component may be added before the oleaginous component is added, dissolved or dispersed in the oleaginous component to be added, added after ail of the oleaginous component is added, or added while the oleaginous component is added. All amount of the water and/or polyvalent alcohol may be added at first, or a part of the addition amount may be added and the remnant amount may be added later.

In particular, when water and. the polyvalent alcohol are used in combination, the biosurfactant and the alkaline substance may be added to all or a part of the water to obtain a first solution, the polyvalent alcohol is mixed with the first solution to obtain a second solution, and the oleaginous component or the remnant water and the oleaginous component are added to the second solution. The gelatinous composition produced by the steps has particularly excellent stability over time.

An example of a use application of the gelatinous composition according to one or more embodiments of the present disclosure may include a skin preparation and a cosmetic preparation. For example, the gelatinous composition can be applied to a basic skin care such as a cream, a lotion, a cleansing gel and a cleansing cream; a cosmetic preparation for makeup, such as a foundation, an eye shadow, a lip color and a lip gloss; a hair care product such as a hair cream, a styling gel and hair wax; a cleaning product such as a shampoo, a hair conditioner, a hand cleanser, a body soap and a cleansing foam.

The present application claims the benefit of the priority date of Japanese patent application No. 2018-3099 filed on Jan. 12, 2018. All of the contents of the Japanese patent application No. 2018-3099 filed on Jan. 12, 2018, are incorporated by reference herein.

### EXAMPLES

For example, the gelatinous composition of one or more embodiments of the present disclosure is prepared as follows, but one or more embodiments of the present disclosure is not restricted to the following Examples.

### Example 1

(A) surfactin sodium salt 0.67 mass %

(B) triethanolamine 0.0167 mass %

(C) glycerin 5 mass %

(D) squalane 91.67 mass %

(E) purified water 2.6433 mass %

A part of the purified water and the triethanolamine were added to the surfactin sodium salt, and the mixture was stirred to obtain 20 mass % surfactin aqueous solution. The glycerin was added to the surfactin aqueous solution, and the mixture was stirred to be mixed. Then the squalane and the remnant purified water were gradually added to the stirred mixture to obtain a gelatinous composition.

### Example 2

A gelatinous composition was produced similarly to Example 1 except that an amount of (B) component was 0.067 mass % and an amount of (E) component was 2.593 mass %.

### Example 3

A gelatinous composition was produced similarly to Example 1 except that 0.0033 mass % of diethanolamine was used as (B) component in place of triethanolamine and an amount of (E) component was 2.6567 mass %.

### Example 4

A gelatinous composition was produced similarly to Example 1 except that 0.01 mass % of diethanolamine was used as (B) component in place of triethanolamine and an amount of (E) component was 2.65 mass %.

### Example 5

A gelatinous composition was produced similarly to Example 1 except that 0.0033 mass % of monoethanolamine was used as (B) component in place of triethanolamine and an amount of (E) component was 2.6567 mass %.

### Example 6

A gelatinous composition was produced similarly to Example 1 except that 0.0083 mass % of diisopropanolamine was used as (B) component in place of triethanolamine and an amount of (E) component was 2.6517 mass %.

### Example 7

A gelatinous composition was produced similarly to Example 1 except that 0.0167 mass % of triisopropanolamine was used as (B) component. in place of triethanolamine and an amount of (E) component was 2.6433 mass %.

### Example 8

A gelatinous composition was produced similarly to Example 1 except that 0.0067 mass % of 2-amino-2-methylpropanediol was used as (B) component in place of triethanolamine and an amount of (E) component was 2.6533 mass %.

### Example 9

A gelatinous composition was produced similarly to Example 1 except that 0.0067 mass % of 2-amino-2-ethyl-1,3-propanediol was used as (B) component in place of triethanolamine and an amount of (E) component was 2.6533 mass %.

### Example 10

A gelatinous composition was produced similarly to Example 1 except that 0.01 mass % of 2-amino 2 -hydroxymethyl-1,3-propanediol was used as (B) component in place of triethanolamine and an amount of (E) component was 2.65 mass %.

### Example 11

A gelatinous composition was produced similarly to Example 1 except that 0.0067 mass % of DL-1-amino 2 propanol was used as (B) component in place of triethanolamine and an amount of (E) component was 2.6533 mass %.

### Example 12

A gelatinous composition was produced similarly to Example. 1 except that 0.0033 mass % of KOH was used as (B) component in place of triethanolamine and an amount of (E) component was 2.6567 mass %.

### Example 13

A gelatinous composition was produced similarly to Example 1 except that 0.01 mass % of KOH was used as (B) component in place of triethanolamine and an amount of (E) component was 2.65 mass %.

### Example 14

A gelatinous composition was produced similarly to Example 1 except that 0.0267 mass % of lysine was used as (B) component in place of triethanolamine and an amount of (E) component was 2.6333 mass %.

### Example 15

A gelatinous composition was produced similarly to Example 1 except that 0.033 mass % of lysine was used as (B) component in place of triethanolamine and an amount of (E) component was 2.627 mass %.

### Example 16

A gelatinous composition was produced similarly to Example 1 except that 0.1167 mass % of histidine was used as (B) component in place of triethanolamine and an amount of (E) component was 2.5433 mass %.

### Comparative Example 1

(A) surfactin sodium salt 0.67 mass %

(C) glycerin 5 mass %

(D) squalane 91.67 mass %

(E) purified water 2.66 mass %

A part of the purified water was added to the surfactin sodium salt, and the mixture was stirred to obtain 20 mass % surfactin aqueous solution. The glycerin was added to the surfactin aqueous solution, and the mixture was stirred to be mixed. Then the squalane and the remnant purified water were gradually added to the stirred mixture to obtain a gelatinous composition.

### Comparative Example 2

A gelatinous composition was produced similarly to Example 1 except that 0.033 mass % of disodium hydrogenphosphate was used as (C) component in place of glycerin and an amount of (E) component was 2.627 mass %.

### Comparative Example 3

A gelatinous composition was produced similarly to Example 1 except that 0.33 mass % of disodium hydrogenphosphate was used as (C) component in place of glycerin and an amount of (E) component was 2.33 mass %.

Test Example 1: Stability Assessment

The gelatinous compositions of the above-described Examples 1 to 16 and Comparative examples 1 to 3 were statically placed at 50° C. for up to 20 weeks, and then the appearances were evaluated on the basis of the following criteria. The results are shown in Table 1 and Table 2.

Stability Evaluation Criteria

Good: gelatinous composition was maintained without separation.

Bad: gelatinous composition was separated as the oleaginous component was separated.

TABLE 1

| Component | | Ex. 1 | Ex. 2 | Ex. 3 | Ex. 4 | Ex. 5 | Ex. 6 | Ex. 7 | Ex. 8 |
|---|---|---|---|---|---|---|---|---|---|
| Surfactin Na | | 0.67 | 0.67 | 0.67 | 0.67 | 0.67 | 0.67 | 0.67 | 0.67 |
| Glycerin | | 5 | 5 | 5 | 5 | 5 | 5 | 5 | 5 |
| Squalane | | 91.67 | 91.67 | 91.67 | 91.67 | 91.67 | 91.67 | 91.67 | 91.67 |
| TEA | | 0.0167 | 0.067 | | | | | | |
| DEA | | | | 0.0033 | 0.01 | | | | |
| Monoethanolamine | | | | | | 0.0033 | | | |
| (2-Aminoethanol) | | | | | | | | | |
| Diisopropanolamine | | | | | | | 0.0083 | | |
| Triisopropanolamine | | | | | | | | 0.0167 | |
| 2-Amino-2- | | | | | | | | | 0.0067 |
| methylpropanediol | | | | | | | | | |
| 2-Amino-2-ethyl- | | | | | | | | | |
| 1,3-propanediol | | | | | | | | | |
| 2-Amino-2-hydroxymethyl- | | | | | | | | | |
| 1,3-propanediol | | | | | | | | | |
| DL-1-Amino-2-propanol | | | | | | | | | |
| KOH | | | | | | | | | |
| Lysine | | | | | | | | | |
| Histidine | | | | | | | | | |
| Purified water | | 2.6433 | 2.593 | 2.6567 | 2.65 | 2.6567 | 2.6517 | 2.6433 | 2.6533 |
| Total | | 100 | 100 | 100 | 100 | 100 | 100 | 100 | 100 |
| pH | | 8.033 | 8.506 | 7.535 | 7.96 | 7.985 | 7.804 | 7.813 | 8.114 |
| Stability | 1W | Good | Good | Good | Good | Good | Good | Good | Good |
| assessment | 4W | Good | Good | Good | Good | Good | Good | Good | Good |
| at 50° C. | 5W | Good | Good | Good | Good | Good | Good | Good | Good |
| | 6W | Good | Good | Good | Good | Good | Good | Good | Good |
| | 8W | Bad | Good | Bad | Good | Good | Good | Good | Bad |
| | 10W | | Bad | | Good | Good | Good | Good | |
| | 12W | | | | Good | Good | Good | Good | |
| | 14W | | | | Bad | Bad | Good | Good | |
| | 18W | | | | | | Good | Bad | |
| | 20W | | | | | | Bad | | |
| Component | | Ex. 9 | Ex. 10 | Ex. 11 | Ex. 12 | Ex. 13 | Ex. 14 | Ex. 15 | Ex. 16 |
| Surfactin Na | | 0.67 | 0.67 | 0.67 | 0.67 | 0.67 | 0.67 | 0.67 | 0.67 |
| Glycerin | | 5 | 5 | 5 | 5 | 5 | 5 | 5 | 5 |
| Squalane | | 91.67 | 91.67 | 91.67 | 91.67 | 91.67 | 91.67 | 91.67 | 91.67 |
| TEA | | | | | | | | | |
| DEA | | | | | | | | | |
| Monoethanolamine | | | | | | | | | |
| (2-Aminoethanol) | | | | | | | | | |
| Diisopropanolamine | | | | | | | | | |
| Triisopropanolamine | | | | | | | | | |
| 2-Amino-2- | | | | | | | | | |
| methylpropanediol | | | | | | | | | |
| 2-Amino-2-ethyl- | | 0.0067 | | | | | | | |
| 1,3-propanediol | | | | | | | | | |
| 2-Amino-2-hydroxymethyl- | | | 0.01 | | | | | | |
| 1,3-propanediol | | | | 0.0067 | | | | | |
| DL-1-Amino-2-propanol | | | | | | | | | |
| KOH | | | | | 0.0033 | 0.01 | | | |
| Lysine | | | | | | | 0.0267 | 0.033 | |
| Histidine | | | | | | | | | 0.1167 |
| Purified water | | 2.6533 | 2.65 | 2.6533 | 2.6567 | 2.65 | 2.6333 | 2.627 | 2.5433 |
| Total | | 100 | 100 | 100 | 100 | 100 | 100 | 100 | 100 |
| pH | | 7.664 | 7.863 | 7.82 | 7.717 | 10.177 | 8.581 | 9.778 | 7.662 |
| Stability | 1W | Good | Good | Good | Good | Good | Good | Good | Good |
| assessment | 4W | Good | Good | Good | Good | Good | Good | Good | Good |
| at 50° C. | 5W | Good | Good | Good | Good | Good | Good | Good | Good |
| | 6W | Good | Good | Good | Good | Good | Good | Good | Good |
| | 8W | Good | Good | Good | Good | Good | Good | Good | Bad |
| | 10W | Good | Good | Good | Good | Good | Good | Good | |
| | 12W | Good | Bad | Good | Bad | Bad | Bad | Bad | |
| | 14W | Good | | Good | | | | | |
| | 18W | Good | | Good | | | | | |
| | 20W | Bad | | Bad | | | | | |

TABLE 2

| | | Comparative | Comparative | Comparative |
|---|---|---|---|---|
| Component | | example 1 | example 2 | example 3 |
| Surfactin Na | | 0.67 | 0.67 | 0.67 |
| Glycerin | | 5 | 5 | 5 |
| Squalane | | 91.67 | 91.67 | 91.67 |
| TEA | | | | |
| DEA | | | | |
| Monoethanolamine | | | | |
| (2-Aminoethanol) | | | | |
| Diisopropanolamine | | | | |
| Triisopropanolamine | | | | |
| 2-Amino-2- | | | | |
| methylpropanediol | | | | |
| 2-Amino-2-ethyl-1,3- | | | | |
| propanediol | | | | |
| 2-Amino-2- | | | | |
| hydroxymethyl- | | | | |
| 1,3-propanediol | | | | |
| DL-1-Amino-2-propanol | | | | |
| KOH | | | | |
| Lysine | | | | |
| Disodium | | | 0.033 | 0.33 |
| hydrogenphosphate | | | | |
| Purified water | | 2.66 | 2.627 | 2.33 |
| Total | | 100 | 100 | 100 |
| pH | | 7.33 | 7.522 | 8.018 |
| Stability assessment | 1 W | Good | Good | Good |
| at 50° C. | 4 W | Bad | Bad | Bad |

As the results shown in Table 1 and Table 2, since the oleaginous component was separated in the gelatinous compositions of Comparative examples 1 to 3 after 4 weeks, the gelatinous compositions do not exhibit preservation stability. On the one hand, the gelatinous compositions containing an alkaline substance of Examples 1 to 16 according to one or more embodiments of the present disclosure provide excellent preservation stability as the gelatinous compositions were maintained for more than 4 weeks.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached

What is claimed is:

1. A gelatinous composition, comprising:
a biosurfactant;
water and/or a polyvalent alcohol;
an oleaginous component; and
an alkaline substance,
wherein the alkaline substance is one or more selected from the group consisting of a hydroxy group-containing amine compound, an alkali metal hydroxide, and a basic amino acid, and
wherein a concentration of the oleaginous component is 50 to 99 mass %.

2. The gelatinous composition according to claim 1, wherein the hydroxy group-containing amine compound is one or more selected from the group consisting of triethanolamine, diethanolamine, monoethanolamine, diisopropanolamine, triisopropanolamine, 2-amino-2-methylpropanol, 2-amino-2-methylpropanediol, 2-amino-2-ethyl-1,3-propanediol, 2-amino-2-hydroxymethyl-1,3-propanediol, and DL-1-amino-2-propanol.

3. The gelatinous composition according to claim 1, wherein the alkali metal hydroxide is one or more selected from the group consisting of NaOH and KOH.

4. The gelatinous composition according to claim 1, wherein the biosurfactant is one or more selected from the group consisting of surfactin, arthrofactin, iturin, and salts thereof.

5. The gelatinous composition according to claim 1, wherein the polyvalent alcohol is one or more selected from the group consisting of glycerin, sorbitol, xylitol, diglycerin, and polyethyleneglycol.

6. The gelatinous composition according to claim 1, wherein the gelatinous composition has a pH of 7.0 to 11.0.

7. A skin preparation, comprising the gelatinous composition according to claim 1.

8. A cosmetic preparation, comprising the gelatinous composition according to claim 1.

9. A method for producing a gelatinous composition,
the method comprising:
dissolving a biosurfactant and an alkaline substance in water to obtain a first solution,
mixing the first solution with a polyvalent alcohol to obtain a second solution, and
adding either an oleaginous component or water and the oleaginous component to the second solution,
wherein the gelatinous composition comprises the biosurfactant, water, the alkaline substance, the polyvalent alcohol, and the oleaginous component,
wherein the alkaline substance is one or more selected from the group consisting of a hydroxy group-containing amine compound, an alkali metal hydroxide, and a basic amino acid, and
wherein a concentration of the oleaginous component is 50 to 99 mass %.

10. The method according to claim 9, wherein the hydroxy group-containing amine compound is one or more selected from the group consisting of triethanolamine, diethanolamine, monoethanolamine, diisopropanolamine, triisopropanolamine, 2-amino-2-methylpropanol, 2-amino-2-methylpropanediol, 2-amino-2-ethyl-1,3-propanediol, 2-amino-2-hydroxymethyl-1,3-propanediol, and DL-1-amino-2-propanol.

11. The method according to claim 9, wherein the alkali metal hydroxide is one or more selected from the group consisting of NaOH and KOH.

12. The method according to claim 9, wherein the biosurfactant is one or more selected from the group consisting of a surfactin salt, an arthrofactin salt, and an iturin salt.

13. The method according to claim 9, wherein the polyvalent alcohol is one or more selected from the group consisting of glycerin, sorbitol, xylitol, diglycerin, and polyethyleneglycol.

14. The method according to claim 9, wherein the gelatinous composition has a pH of 7.0 to 11.0.
